(19) United States
(12) Patent Application Publication
(10) Pub. No.: US 2025/0041252 A1
(43) Pub. Date: Feb. 6, 2025

(54) POWDERS FOR INHALATION AND PRODUCTION PROCESS THEREOF

(71) Applicants: UNIVERSITA' DEGLI STUDI DI PARMA, Parma (IT); UNIVERSITA' DEGLI STUDI DI FIRENZE, Firenze (IT); AZIENDA OSPEDALIERO-UNIVERSITARIA CAREGGI, Firenze (IT)

(72) Inventors: Ruggero Bettini, Parma (IT); Francesca Buttini, Parma (IT); Giovanni Fontana, Firenze (IT); Federico Lavorini, Firenze (IT)

(73) Assignees: UNIVERSITA' DEGLI STUDI DI PARMA (50%), Parma (IT); UNIVERSITA' DEGLI STUDI DI FIRENZE (25%), Firenze (IT); AZIENDA OSPEDALIERO-UNIVERSITARIA CAREGGI (25%), Firenze (IT)

(21) Appl. No.: 18/718,808

(22) PCT Filed: Dec. 15, 2022
(86) PCT No.: PCT/IB2022/062286

§ 371 Date: Jun. 12, 2024

(30) Foreign Application Priority Data
Dec. 17, 2021 (IT) 102021000031637

(51) Int. Cl. A61K 31/167; A61K 9/00; A61K 9/16; A61P 11/14

(52) U.S. Cl. CPC A61K 31/167; A61K 9/0075; A61K 9/1652; A61K 9/1682; A61P 11/14

(57) ABSTRACT

Pharmaceutical compositions in the form of dry powder for inhalation comprising a local anesthetic such as procaine, chloroprocaine, lidocaine, prilocaine, mepivacaine, bupivacaine, etidocaine, ropivacaine, and tetracaine or salts and/or solvates thereof and a hydrophilic biocompatible polymer, in particular hyaluronates, are described. The compositions of the invention are useful for the treatment of the cough.

### FIELD OF THE INVENTION

The present invention relates to a combination of a local anesthetic (LANE) drug with a hydrophilic biocompatible polymer to be administered as a dry powder formulation for inhalation by means of a dry powder inhaler (DPI) for the treatment of the chronic cough and the prevention of the cough induced by pharmacological treatments, as well as for the treatment of the acute cough, for example during airways viral infections or in post-infective cough. Further, the invention is directed to a process for the preparation thereof.

### BACKGROUND ART

The inhalation therapy is one of the oldest, but also most effective, approaches to the therapy of airways diseases. To date, it is well known that the most effective and secure way to treat lungs is administering drugs directly onto the airways thus giving rise to a low systemic exposure and a rapid onset of the pharmacological response.

The cough is an important defensive mechanism of the respiratory apparatus, and it is the most frequently observed sign during diseases interesting this district of the organism. Acute cough is meant when the disorder persists less than 4 weeks, while chronic cough is meant when the disorder persists beyond eight weeks (Morice AH et al., Eur Respir J 2020 55: 1901136).

The chronic cough affects about 10% of the general adult population, and it constitutes an important health problem due to the negative impact on life quality of affected people and for possible complications. In about 25% of the cases, the chronic cough is defined as “inexplicable” since it remains without explanation even if the patient has been subjected to numerous diagnostic investigations, or “refractory” since insensible to various treatments. This kind of cough, which we prefer to call “idiopathic,” represents a difficult challenge from the clinical point of view (Song, W.-J et al., Allergy Asthma Immunol Res 2016, 8 (2), 146-10). To date, no authorized product for inhalation exists for this indication. Anyway, it is well known that local anesthetics (hereinafter referred to as LANEs) could act as peripheral anti-cough drugs blocking neuronal sodium channels, reducing the excitability, and therefore the cough reflex (Karlsson JA Bull Eur Physiopathol Respir. 1987; 23 Suppl 10: 29s-36s; Dicpinigaitis PV et al. Pharmacol Rev 2014; 66:468-512).

The off-label use of LANEs for the local treatment by nebulization of solutions represents a not rare clinical practice. A retrospective study on adults receiving a prescription and a nursing instruction for the administration of a nebulized lidocaine solution for the chronic cough between 2002 and 2007 (Lim, K. G. et al., Chest 2013, 143 (4), 1060-1065) demonstrated that adults tolerate the self-administration of nebulized lidocaine for the chronic cough difficult to control. Severe adverse effects have not been observed, anyway most patients disliked the treatment for the negative effects connected to the administration by nebulization, such as, unpleasant taste, throat or oral cavity irritation, loss of sensorial function.

Recently, a clinical trial compared the efficacy of lidocaine administration in reducing the effects related to the refractory chronic cough, showing the higher efficacy of spray formulations with respect to nebulization (Rayid Abdulqawi et al., The Journal of Allergy and Clinical Immunology: In Practice. 2021, 9(4) 1640-1647).

Beside these issues, the deposition of a LANE in a liquid form (for example, nebulized droplets) can generate the risk of drug deposition in the lung deep portion resulting in the adsorption and systemic distribution thereof. This can cause CNS excitation followed by depression which represent the most common outcomings of toxicity by local anesthetics (Covino, B. G. et al., J. Dent. Res. 1981, 60 (8), 1454-1459).

To date, no solid particle formulations consisting of a combination of LANE and a polymer suitable for deposition on the conductive part of the respiratory tree are described.

An excipient-free inhalable formulation of lidocaine is described for asthma treatment and to reduce the need of corticosteroids in asthmatic patients (WO 2006/6181). In this case, the formulation consists of a drug without any specific production process. The same assignee claimed a pure formulation of benzyl phosphate or a benzyl phosphate prodrug of a corticosteroid, lidocaine, or a related local anesthetic composition for administration by aerosol to inhibit the inflammation in the lungs of asthmatic patients (WO 2005/063777). Similarly, a N-oxide prodrug of a local anesthetic for inflammation associated to bronchitis and COPD was described (WO 2005/044233).

Other two applications of an inhalable local anesthetic are available: one as an anti-migraine product in the form of a liquid aerosol (US 20040184999), while the other is a formulation in the form of a pressurized metered-dose inhaler (US 19975679325).

Therefore, there is still a significative need of a more effective and more secure LANE therapy for the treatment of the cough, in particular the chronic cough.

In recent years, in scope of inhalation therapy, possible alternatives to nebulization were studied and DPIs were proposed as a promising strategy due to the possibility to easily administer a sufficient amount of micronized powder containing the therapeutic dose. A DPI comprises a formulation aerosolized by a passive inhaler device activated by the act of inhalation from the patient. DPIs can disperse a high amount of drug in a unique and rapid act of inhalation (20 mg in few seconds); if a higher amount is required, the dose can be inhaled by subsequent inhalations (Buttini, F et al., Int J Pharm 2018, 548 (1), 182-191).

In general, the development of powders for inhalation requires the overcoming of a well-known paradox: since the active ingredient (API) should have a very small particle size to reach the lungs but at the same time must show favorable technological properties (flowability, packing, re-dispersion) to allow the release of the accurate dose and aerosol of the aerosolized drug. This consideration becomes more important when the API dose is high. The technological properties of a powder consisting of the API alone are generally poor and not suitable for administration by inhalation. In fact, particles of suitable size for administration by inhalation are generally obtained subjecting the API to a micronization process, usually in fluidized jet mills. These processes imply an increase of the energetic content of the particles and of their reactivity and induce the creation of highly cohesive surfaces. All these aspects are extremely unfavorable for the administration by inhalation. In the case wherein the API is a LANE, in particular a LANE with a medium duration of action, such as lidocaine, the low melting point is a further aspect inducing issues and instability during the manufacturing processes (above all micronization) which often imply an increased temperature of the treated material.

Contrary to what happens for drugs administered to lung which have bronco-alveolar region as site of action, in the specific case of the cough, and in particular the chronic and refractory cough, the LANE administration has to maximize the deposition at the level of the conductive portion of the respiratory tree where the highest excitability occurs and from which the cough reflex originates. From the point of view of the delivery technologies, the deposition in the conductive portion, namely of the 0-15 generation according to the Weibel model (Weibel ER. 1963. Morphometry of the human lung. Berlin: SpringerVerlag), implies the overcoming of a substantially different and new issue with respect to those related to the administration of drugs which must be deposited at bronchoalveolar level. In fact, the conductive region has a lower number of ramifications inducing a flow modification resulting in the loss of particle moment and it is covered by a ciliated epithelium physiologically assigned to the capture and removal of the particles transported by the inspiratory flow. This generates a lower possibility of particle deposition in the site of action by formulations designed to be deposited in the deepest parts of the lung, as the adhesive mixtures (De Boer, AHD et al., Expert opinion on drug delivery 14, 499-512).

In general, it is possible to develop particle engineering strategies to control the physical shape and control the size and the morphology in order to optimize the performances of DPIs with respect to the maximization of the depositable fraction in the deepest part of the lung and the enhancing of the flow properties (Buttini, F et al., J Control Release 2012, 161 (2), 693-702). Anyway, in the case of the administration of drugs that must act at the level of the conductive portion of the respiratory tree, for the above-mentioned reasons, the issue of the deposition in the site of action of a powder for inhalation cannot be entirely solved with the simple optimization of the particle size.

However, there is no doubt that it would be highly advantageous to have LANE-based medicinal products suitable for administration by inhalation, allowing a rapid and easy drug administration giving rise, at the same time, to a high deposition of LANE on the target site. From this point of view, a formulation is needed favoring the deposition of the medicament in the respiratory apparatus area wherein the cough receptors are located. The quickly adapting cough receptors or irritative receptors are principally on the rear wall of trachea, pharynx, and on the carina of the trachea, the point where the trachea branches into the main bronchi. The receptors are less numerous in the distal airways and absent beyond the respiratory bronchioles. Therefore, the more suitable aerodynamical size for the particles to direct the medicament in this area is comprised between 6 and 12 microns.

The above-described issues are solved by the combination of the present invention and use thereof.

### SUMMARY OF THE INVENTION

In a first aspect, the invention has as an object pharmaceutical compositions in the form of a dry powder for inhalation comprising a local anesthetic and a hydrophilic biocompatible polymer.

Advantageously, said powder is in the form of dry micro-particles obtained by spray-drying to be administered by inhalation by means of a dry powder inhaler (DPI). After aerosolization by means of an inhalation device, said micro-particles typically have a mass median aerodynamic diameter comprised between 4.0 and 6.0 microns and are characterized by a fine particle fraction not higher than 35% by mass.

Also, the invention relates to a process for the preparation of the micro-particles and comprising the following steps:
i) selection of a hydrophilic biocompatible polymer and dissolution thereof in a suitable solvent at a suitable concentration.
ii) selection of a LANE drug and dissolution thereof in water at a suitable concentration.
iii) addition of the solution of step ii) to the solution of step i) keeping under stirring the resulting solution.
iv) spray-drying of the solution of step iii) using a suitable spray-drying apparatus.
v) collection of the resulting powder in the form of particles; and
vi) optional micronization of said particles.

In a preferred embodiment, particles with the diameter suitable for use according to the invention are obtained using a spray-dryer nozzle having a diameter comprised between 0.7 mm and 3 mm.

The invention further relates to a dry powder inhaler filled with the aforementioned dry powder pharmaceutical formulation and a kit comprising said dry powder compositions and a dry powder inhaler.

The invention has as a further object said compositions for use as a medicament, in particular for the treatment of the cough.

### DEFINITIONS

In the present invention:
The term “LANE” refers to therapeutical substances, belonging to the class of local anesthetics having advantageously a water solubility of at least 0.5% w/v according to the definition and procedures of determination thereof as reported in the U.S, European, and British Pharmacopoeias.
Local anesthetics are drugs that can be classified based on their intrinsic anesthetic potency and activity duration, according to the literature. For example, procaine and chloroprocaine are drugs with a relatively low potency and with a short action duration. Lidocaine, mepivacaine, and prilocaine represent agents with an intermediate potency and action duration. Tetracaine, bupivacaine, and etidocaine are very potent agents with a prolonged action.
The cough is a physiological defense mechanism of the respiratory apparatus with the aim to keep airways free from excess secretions and accidentally inhaled foreign material. It can be voluntary and involuntary. In the medical literature several guidelines for categorizing the cough exist (Irwin RS and Madison JM. New England Journal of Medicine 2000, 343 (23, 1715-1721; Morice AH, et al Eur Respir J 2020 55: 1901136; DOI: 10.1183/13993003.01136-2019). The cough lasting less than 4 weeks is generally considered as “acute” and viral infections of the upper respiratory tract are the most common cause of the acute cough. The cough lasting between three and eight weeks is classified as subacute and the cough exceeding eight weeks is defined as chronic.
As polymer molecular weight is meant the average molecular weight (M_{w}) by mass.
The term mucoadhesive polymer defines a hydrophilic polymer able to interact with the mucous layer covering the respiratory epithelium through weak and reversible binds (S. Roy, et al. (2009) Polymers in Mucoadhesive Drug-Delivery Systems: A Brief Note, Designed Monomers and Polymers, 12:6, 483-495).
The term “micronized” relates to a powder showing 90% of particle size distribution less than 10 μm.
The term “gross” relates to a substance having a size of a few hundred microns.
In general terms, the particle size is quantified measuring a characteristic diameter of the equivalent sphere, known as volume diameter, by laser light diffraction.
The particle size can be quantified also measuring the diameter of the mass by a suitable instrument such as the sieve analyzer.
The volume diameter (VD) is related to the mass diameter (MD) through the particle density (assuming a density independent from the particle size).
In the present description, the particle size of active ingredients and of the fine particle fraction is expressed in terms of volume diameter, while the one of gross particles is expressed in terms of mass diameter.
Particles have a normal or log-normal distribution defined in terms of volume or mass median diameter (VMD or MMD, respectively) corresponding to the volume or mass diameter of 50 percent in volume or mass of the particles and, optionally, in terms of volume or mass diameter of 10% and 90% of the particles, respectively.
Another common approach to define the particle size distribution consists of using three values: i) the median diameter d(0.5) which is the diameter dividing the distribution in two equal parts; ii) d(0.9) and iii) d(0.1), corresponding to 90° and 10° percentile of the distribution, respectively.
In general terms, particles having the same or similar VMD or MMD can have a different particle size distribution, and in particular a different width of the distribution represented from the values d(0.1) and d(0.9).
Upon aerosolization, the particle size is expressed as mass aerodynamic diameter (MAD), while the particle size distribution is expressed in terms of mass median aerodynamic diameter (MMAD) and geometrical standard deviation (GSD). MAD depends on the ability of the particles to be transported in suspension in an air flow. MMAD corresponds to the mass aerodynamic diameter of 50 percent by weight of the particles.

The expression “breathable fraction” refers to the percentage of active particles reaching the lungs in a patient, i.e., the mass of particles having an aerodynamic diameter less than 5 μm.
MMAD, GSD, and breathable fraction are assessed using a suitable impactor as Andersen Cascade Impactor (ACI), Multi Stage Liquid Impinger (MLSI), or Next Generation Impactor (NGI), according to procedures reported in the common Pharmacopoeias, in particular in the European Pharmacopoeia 10^{th} Edition.
The breathable fraction is calculated from the percentage ratio between the fine particle mass (also referred to as fine particle dose) and the delivered dose representing the mass of active ingredient emitted from the device upon activation.
The delivered dose, meant as the measured dose amount emitted from the inhaler following to an inhalation act, is calculated from the cumulative deposition in the impactor apparatus, while the fine particle mass is calculated by the deposition of the particles with aerodynamic diameter <5.0 μm.
Also, the delivered dose is assessed using a dose unit sampling apparatus (DUSA) according to procedures reported in the common Pharmacopoeias, in particular in the European Pharmacopoeia 10^{th} Edition.
As metered-dose (MD), is meant the powder mass loaded in the inhaler and intended to be released (as a whole or in part) for effect of the device activation.
The “emitted fraction” (EF) is the ratio between the emitted dose and the measured dose.
The fraction of not-breathable particles is calculated from the percentage ratio between the mass of not-breathable particles (i.e., with an aerodynamic diameter >5 μm) and the delivered dose.
The not-breathable particle mass (NB-PM) is defined as the drug amount with aerodynamic diameter higher than 5 μm, obtained as the difference between the emitted dose and the fine particle mass, and expressed in mg.
“Treatment” of the cough means reducing the frequency of the coughing events and/or reducing the severity of the coughing events (with respect to the not-treated condition). These terms refer both to the preventive treatment and treatment of the ongoing coughing episodes.
A “therapeutically effective amount” of a substance refers to an amount leading to a clinically significative reduction of the frequency or severity of the coughing events.

### DETAILED DESCRIPTION OF THE INVENTION

The invention is directed to a combination of a local anesthetic drug (LANE) with a hydrophilic biocompatible polymer (HBP), LANE and HBP being present in a pre-established ratio.

Advantageously, the LANE can be any active ingredient belonging to the pharmacological class of anesthetics. More advantageously, the LANE has a water solubility in standard conditions (15-25° C., 1 atm) of at least 0.05% w/v.

For example, LANE drugs can be chosen among LANEs with a short, intermediate, and long action duration such as procaine, chloroprocaine, lidocaine, prilocaine, mepivacaine, bupivacaine, etidocaine, ropivacaine, and tetracaine and/or salts and/or solvates thereof. If one of these compounds has chiral centers, they can be used in an optically pure form, or can be present as diastereomeric mixtures or racemic mixtures.

Advantageously, the LANE can be used in the form of a free base or a pharmaceutical acceptable salt as hydrochloride and hydrobromide. In a preferred embodiment, the LANE is lidocaine in the form of hydrochloride salt.

Advantageously, the hydrophilic biocompatible polymer (HBP) can be selected from the group consisting of safe and pharmaceutical acceptable substances as hyaluronic acid (HA) salts, preferably the sodium salt, water-soluble cellulose derivatives, polyethylene glycols, polyvinyl pyrrolidones, polyvinyl alcohols, or any mixtures thereof.

Preferably, the hydrophilic biocompatible polymer is sodium hyaluronate (SH) of molecular weight comprised between 15 and 1500 kDa, preferably between 15 and 200 kDa, more preferably between 20 and 140 kDa, and even more preferably between 20 and 100 kDa.

More advantageously, the LANE is present in a percentage by weight of the combination comprised between 1 and 90%, preferably between 10 and 50% by weight of the combination. Preferably, the percentage by weight of HA ranges from 90 to 50% w/w, more preferably from 80 to 70% w/w.

Advantageously, the combination of the invention is in the form of micro-particles characterized by a defined particle size.

Expressed as volume diameter, the particle size distribution of micro-particles should fulfil the following parameters: d(0.1) comprised between 2.0 and 5.0 microns, d(0.5) comprised between 5.0 and 9.0 microns, and d(0.9) comprised between 11 and 18 microns.

When expressed as aerodynamic diameter, the mass median value is comprised between 4.0 and 6.0 microns.

When aerosolized by means of a common DPI, micro-particles show a Fine Particle Fraction (FPF) (<5 microns) comprised in a range of 35-30%, preferably in the range of 25-20%, more preferably in the range of 15-20%.

The advantages of the dry powder compositions of the invention derive from the obtaining of engineered micro-particles comprising a LANE in combination with a hydrophilic biocompatible polymer, suitable for inhalation, and able to satisfy the requirements deriving from the particular difficulties related to the deposition of the medicament in the conductive tract of the respiratory tree.

The powder of the invention consists of flowable particles of LANE able to be effectively delivered from an inhaler device and deposited on the mucosa layer covering the ciliated epithelium of the conductive airways due to a defined size comprised between 2 and 18 microns, and preferably between 5 and 14 microns, and even more preferably between 5 and 12 microns.

The presence in the particles of a mucoadhesive hydrophilic biocompatible polymer favors their adhesion to the wet epithelium and a localized and prolonged over time release of LANE.

The micro-particles of the invention are stable both from the chemical and physical point of view.

The compositions of the invention show a homogeneous distribution of the active ingredient in the powder and the possibility to be delivered at high doses: the maximum dosage administrable through one or more inspiratory acts from the patient is 100 mg, preferably 80 mg, and even more 40 mg of powder.

Even if not strictly required, the powder formulations according to the invention can further comprise gross and/or fine particles of a pharmaceutical acceptable inert excipient such as lactose, preferably alpha-lactose monohydrate, trehalose, mannitol, raffinose.

The present invention allows the preparation of a powder of HA particles effectively aerosolizable by adding a LANE. According to the invention, the powder dose delivered from the inhaler is >70% by weight of the loaded dose and preferably >85% by weight of the loaded dose.

The composition of the invention can be loaded in rigid capsules, blisters, or reservoir inhaler without adding any flow excipient or diluent.

The micro-particles of the invention are consisting of a matrix of a HBP wherein the LANE molecules are trapped giving rise to an amorphous, kinetically stable for a pharmaceutical acceptable time, semicrystalline or crystalline stable structure. Therefore, the compositions of the invention can be aerosolized and inhaled using a dry powder inhaler.

The particle size allows for their deposition on the area of the respiratory tree (larynx, trachea, large bronchi) where the receptors for the cough stimulus are highly concentrated; this characteristic allows to maximize the therapeutic effect, minimizing the systemic adsorption and the side effects related to it.

Further, the HBP-LANE micro-particles of the invention release the active ingredient progressively in a prolonged period of time.

The present invention further relates to a process for preparing said engineered micro-particles acting on the choice of the polymer type, its molecular weight, and on the polymer: drug ratio.

In particular, the process of the invention is performed by spray-drying.

Typically, spray-drying is performed by nebulizing a solution containing the solutes to be dried in a pre-heated drying chamber wherein the small droplets of the solution are subjected to a hot gas flow at a controlled temperature and converted in particles of powder. The obtained powder goes through a powder/gas separator, for example a cyclone wherein it loses kinetic energy so to be collected in a suitable container.

The adjustable parameters to obtain a powder with well-defined characteristics are: i) the nebulizer type; ii) the temperature of the incoming gas used to dry the material sprayed in the drying chamber (hereinafter referred to as input temperature); iii) the gas flow rate, and iv) the flow rate of the supply solution (hereinafter referred to as supply flow rate).

Said spray-drying process comprises the following steps:
i) selection of a suitable hydrophilic biocompatible polymer (HBP) and dissolution thereof in a suitable solvent at a suitable concentration.
ii) selection of a LANE drug and dissolution thereof in water at a suitable concentration.
iii) addition of the solution of step ii) to the solution of step i) keeping under stirring the resulting solution.
iv) spray-drying of the solution of step iii), using a suitable spray-drying apparatus.
v) collection of the powder obtained in the form of particles; and
vi) optional micronization of said particles.

Advantageously, in step i) the solvent is water or a hydroalcoholic solution containing ethanol as co-solvent in a concentration comprised between 0.1 and 99.9% v/v.

Preferably step i) is performed in water at room temperature and under stirring wherein HBP is comprised between 1 and 6% w/v.

Advantageously, even in step ii) the solvent is water or a hydroalcoholic solution containing ethanol as co-solvent in a concentration comprised between 0.1 and 99.9% v/v.

The HBP type in step i) and its molecular weight must be chosen by the person skilled in the art based on the specific qualitative composition, the drug type, and the drug content. In a preferred embodiment of the invention HA is selected as polymer.

Preferably, step ii) is performed at room temperature and under stirring wherein the concentration of the drug LANE is comprised between 0.6 and 10%, and preferably between 1 and 6% w/v.

The solution of step iii) should have a solute concentration comprised between 0.5 and 6% w/v, and preferably comprised between 3-5% w/v.

The concentration of LANE expressed as a free base in the solution obtained from step iii) is comprised between 10 and 50%, and preferably between 20 and 40% on the solute weight.

Advantageously, the resulting solution from step iii) is kept under stirring for at least 50 rpm for 10 minutes.

Step i) and step ii) can be combined and the solution can be prepared combining all the components of the two steps and heating their content at a temperature comprised between 25 and 50° C., preferably between 30 and 44° C., and more preferably between 37 and 42° C.

In step iv), the HBP-LANE solution is then spray-dried with suitable parameters, such as:
input temperature, preferably between 110° C. and 160° C., preferably between 115° C. and 150° C., and even more preferably between 120° C. and 140° C.;
flow rate of the supply solution between 1.0 and 10.0 mL/min, preferably between 1.5 and 5 mL/min, and even more preferably between 2 and 4 mL/min;
solute concentration of the solution between 1 and 7% w/v, preferably between 2 and 6% w/v, and even more preferably between 3 and 5% w/v;
nebulization gas flow rate between 135 and 820 L/h, preferably between 300 L/h and 750 L/h, and even more preferably between 473 and 670 L/h;
drying gas flow rate between 20 and 40 m³/h, preferably between 30 and 38 m³/h, and even more preferably between 33 and 35 m³/h.

In step v) the particles can be collected according to known methods.

In step vi) the collected particles can be optionally micronized according to known methods. Anyway, according to the invention, the micro-particles of the desired size can be obtained without further micronization using a spray-dried nozzle having a diameter comprised between 0.7 mm and 3.0 mm, preferably between 1.0 mm and 2.5 mm, and more preferably between 1.4 mm and 2.0 mm.

The solution of step iii) is prepared adding the LANE solution to the HBP one, anyway, the one skilled in the art can choose different preparation methodologies and conditions based on his/her own knowledges.

In step iii), in particular in the case of lidocaine powders and 50-90% by weight of HA, it was found that the preferred molecular weight of HA is in the range 20-100 kDa ensuring the formation of micro-particles with a totally amorphous structure which is maintained for a pharmaceutically acceptable time. For higher HA molecular weights, in the order of 750-1000 kDa, the obtained micro-particles can show a partially crystalline structure observed, for example, by polarized light microscopy, attributable to the not complete molecular dispersion of lidocaine between the HA polymeric chains.

The particle size distribution of the powder is significantly influenced by the diameter of the nozzle used for the spray-drying process. Using a 1.4 mm diameter, d(v, 10) it was of 2.4-2.8 microns and d(v, 90) of 11.0-14.0 microns. These particles populations had a MMAD of 3.8-4.1 microns and a FPF of 34% indicating a percentage of particles of (66%) in the range 5-14 microns. When a diameter higher than 2.0 mm was used, the micro-particles showed a MMAD of 5.7-6.0 microns and a FPF of 14% indicating a high percentage of particles (86%) in the range 5-17 microns. This observation is confirmed by laser diffraction analysis revealing a d(v, 10) of 3.3-4.6 microns and d(v, 90) of 14.2-17.0 microns.

This peculiar characteristic causes that the powder deposits in particular on the respiratory tree areas (larynx, trachea, large bronchi) wherein the cough receptors are highly located, minimizing the deposition in the oral cavity responsible for unpleasant and/or severe side effects such as sensibility loss or even suffocation upon food and drink intake.

A further advantage of the invention is represented by the fact that the polymer, for example HA, acts a control element of the LANE release, prolonging its deliverance in the action site for several hours with respect to the dissolution of the LANE as it is, which, instead, takes place in few minutes. This aspect is favorable to obtain a prolonged cough suppression.

Finally, the HBP mucoadhesive ability favors the particles adhesion to the mucosa covering the epithelium of the conductive tract of the respiratory tree avoiding that the particles flow in the low airways where the systemic adsorption is higher.

The therapeutic amount of the combination can vary within wide limits according to the active substance nature, the type and severity of the condition to be treated, and the condition of the patient in need of treatment.

In particular, the active substances are added to the compositions of the invention carefully following the solution preparation and the drying process.

An excellent distribution uniformity of the active ingredient is obtained when said active ingredient has a drug content within 5% with respect to the theoretical value. The content uniformity determination must be performed by those skilled in the art using a validated analytical method.

The composition of the invention can be used with any dry powder inhaler: i) mono-dose inhalers (unit dose), for the administration of subdivided single doses of the active ingredient; ii) metered multi-dose inhalers or reservoir inhalers pre-loaded with active ingredient amounts enough for longer treatment cycles.

The composition of the invention can be administered to a patient at an established frequency as prescribed by a clinician, for example in single or multiple doses, typically once, twice, or several times per die. Alternatively, the compositions of the invention can be administered by a caregiver or self-administered by the patient according to need.

The composite dry powder amount to be inhaled depends on the active ingredient concentration in the spray-dried powder. For example, 16 mg of powder will be required to administer 8 mg of LANE from a mixture HBP:LANE 50:50 w/w, or 40 mg of powder will be required to administer 8 mg of LANE from a mixture HBP:LANE 80:20 w/w. Preferably, the drug amount will be administered in a single inhalation act (the entire powder dose in few seconds), but in a particular embodiment of the invention, if a higher powder amount is required, the dose can be inhaled through several consecutive inhalations by means of the same inhaler.

The compositions of the invention are effective for the treatment of the acute, subacute, or chronic cough of various etiology, for example the cough associated to asthma or due to the administration of another medicine, such as an ACE-inhibitor, or any medicine used to trat asthma or chronic obstructive pulmonary disease.

The invention is illustrated in detail by the following Examples.

Example 1. Production of spray-dried powders of lidocaine hydrochloride-HA of molecular weight comprised between 20-50 kDa.

The optimization was centered on three process parameters, i.e., solute concentration in the solution to be dried, supply flow rate, and nozzle diameter. Each of three variables was considered at two levels applying a complete 2-levels factorial design using the software Minitab® 17 (Minitab Inc., State College, PA, USA) performing a total of 8 experiments (2³).

The following dried powder quality attributes were assessed:
Residual moisture content of the dried powder
Particle size as dv50
Not-breathable fraction

The composite spray-dried powder contained lidocaine hydrochloride (ACEF, Fiorenzuola, Italy) 20% w/w, and HA 20-50 kDa (Prymalhyal 50, 20-50 kDa, Givaudan, France) 80% w/w as mucoadhesive polymer.

The powders were produced using a Mini spray dryer B290 apparatus (Büchi, Switzerland) keeping fixed the following process parameters: air flow rate (600 L/h), input temperature (130° C.), and flow rate in aspiration (35 m³/h). With such a condition, the output temperature was about 70° C.

The mass median diameter, Dv50, was determined by laser diffraction as described in Example 2.

The water content of the spray-dried powders was measured by thermogravimetric analysis (TGA) using a TGA-DSC 1 STAR^{e} system equipment (Mettler Toledo, USA). Approximately, 5 mg of each spray-dried powder were subjected to a heating program from 25 to 130° C. with a heating rate of 10° C./minute under a flow of 50 mL/minute of dry nitrogen.

The residual moisture content was represented by the percentage weight loss occurring between 25 and 110° C.

The not-breathable fraction was assessed in vitro using a Fast Screening Impactor (FSI, Copley Scientific, UK). This equipment uses two segregation stages: the first named CFC, where particles with an aerodynamic diameter higher than 5 μm are deposited, and one named FFC collecting particles with an aerodynamic diameter less than 5 μm. The FSI consists of an induction port (IP), a CFC filled with 10 mL of a solution methanol-water 70:30 v/v, acting as liquid trap for particles with aerodynamic diameter >5 μm and a FFC equipped with a glass-fiber filter (A/E Type, Pall Corporation, USA). After completing the assembly according to the instructions provided by the manufacturer, the FSI was connected to a vacuum pump VP 1000 (Erweka, Germany) and the air flow of 60 mL/min was recorded through the impactor, measured by a flowmeter (model 3063, TSI, USA). The powder aerosolization was performed by means of a device for dry powders RS01 (Plastiape, Italy) loaded with a hypromellose capsule format #3 (Qualicaps, Spain) containing 20 mg of powder. After deposition, the powder amount in each stage, in the device, and in the capsule, was collected by high performance liquid chromatography (HPLC) using a defined solvent volume. The analysis was performed in triplicate.

The not-breathable fraction was calculated dividing the amount deposited in the CFC stage by the total powder amount emitted from the device, corresponding to the sum of the amount deposited on CFC and FFC.

Table 1 summarizes the process parameters and the corresponding quality attributes obtained for the lidocaine hydrochloride-HA powders.

TABLE 1
Process variables and corresponding quality attributes (average value ±
standard deviation) of the powders containing lidocaine hydrochloride 20% w/w and
HA (20-50 kDa) 80% w/w obtained with a complete factorial design 2³.

| | Process parameters | | | Quality attributes | | |
|---|---|---|---|---|---|---|
| | Solute | Supply flow | Nozzle | | Residual | Not-breathable |
| | concentration | rate | diameter | | moisture | fraction |
| Powder | (% w/v) | (mL/minute) | (mm) | Dv50 | (% w/w) | (%) |
| #1 | 5 | 2.00 | 2.0 | 8.1 ± 0.2 | 7.4 | 81.3 ± 2.9 |
| #2 | 3 | 2.00 | 2.0 | 8.5 ± 0.2 | 8.26 | 73.0 ± 2.0 |
| #3 | 5 | 4.00 | 2.0 | 8.9 ± 0.1 | 8.6 | 73.0 ± 0.9 |
| #4 | 3 | 4.00 | 2.0 | 7.3 ± 0.2 | 8.69 | 72.8 ± 3.8 |
| #5 | 5 | 4.00 | 1.4 | 6.6 ± 0.1 | 8.91 | 58.2 ± 2.4 |
| #6 | 5 | 2.00 | 1.4 | 6.9 ± 0.1 | 7.74 | 57.9 ± 1.0 |
| #7 | 3 | 2.00 | 1.4 | 5.8 ± 0.4 | 8.01 | 51.2 ± 3.6 |
| #8 | 3 | 4.00 | 1.4 | 5.4 ± 0.0 | 8.00 | 53.3 ± 0.3 |

A statistical analysis (ANOVA) was performed with Minitab® 17 software and the effects of process parameters and quality attributes were analyzed with linear regression models.

The regression equations showed that the nozzle diameter had a statistically more important effect on the distribution of the particle sizes and on the not-breathable fraction: increasing the nozzle diameter, the particle size of the spray-dried powders and the not-breathable dose increase.

Example 2. Particle size distribution and drug content of the spray-dried lidocaine hydrochloride-HA (20-50 kDa) powders.

The particle size distribution of the spray-dried lidocaine-HA powders prepared according to Example 1, was assessed by laser diffraction using the Spraytech instrument (Malvern, UK).

About 10 mg of spray-dried powder were dispersed in a polysorbate 80 (SPAN® 80, sigma Aldrich, USA) solution in cyclohexane wherein the lidocaine hydrochloride is insoluble (powder concentration 0.1% w/v) and placed in an ultrasound bath for 5 minutes before the diffractometric measurement.

Each measurement was performed in triplicate. The obscuration of the laser beam was kept between 8 and 10%.

The particle size was expressed as diameter of the equivalent sphere in volume, i.e., dv10, dv50, and dv90.

The drug content and powder homogeneity were assessed by HPLC using a LC Agilent 1200 instrument (Agilent Technologies, USA). The analysis was performed on 6 samples prepared weighting 25 mg of powder dissolved in 10 mL of water. Three replicates were performed for each sample.

The determination of the content uniformity was performed using a validated analytical method and the following chromatographic conditions:
Colum: C18 Agilent® Eclipse (5 μm, 4.6×150 mm, Waters Corp, USA);
Wavelength: 230±2 nm;
Column temperature: 25° C.;
Flow: 1 mL/minute;
Mobile phase: phosphate buffer solution: methanol (25:75 v/v).

The phosphate buffer was prepared dissolving 1.38 g of anhydrous NaH₂PO₄ in 500 mL of ultrapure water, then the pH was adjusted to 8 by adding few drops of a 1 M/40 g/L NaOH aqueous solution. The solution was filtered with a 0.45 μm PTFE filter. The drug retention time was 3±1 minutes.

The drug content was homogeneous in all samples and within 5% w/w with respect to the theoretical value. The variation coefficient, VC, (calculated as percentage of the ratio between standard deviation and average value on five measurements) was less than 2.5%. The result indicates that the spray-drying technique is suitable to prepare lidocaine hydrochloride-HA particles in an established combination.

TABLE 2
Particle size distribution and drug content (average values ±
standard deviation) of the powders produced according to Example 1.

| | | | | | Lidocaine | |
|---|---|---|---|---|---|---|
| | | | | | content | |
| | | | | Lidocaine | % w/w of | |
| | | | | content | the | |
| | | | | % w/w of | theoretical | VC |
| Powder | Dv₁₀ | Dv₅₀ | Dv₉₀ | the powder | value | (%) |
| #1 | 4.7 ± 0.1 | 8.1 ± 0.2 | 14.5 ± 0.5 | 20.1 ± 0.4 | 100.4 ± 2.1 | 2.1 |
| #2 | 3.6 ± 0.1 | 8.5 ± 0.2 | 15.9 ± 0.4 | 19.9 ± 0.4 | 99.5 ± 2.0 | 2.0 |
| #3 | 4.0 ± 0.0 | 8.9 ± 0.1 | 17.6 ± 0.4 | 20.1 ± 0.1 | 100.7 ± 0.6 | 0.6 |
| #4 | 3.3 ± 0.0 | 7.3 ± 0.2 | 14.2 ± 0.5 | 20.5 ± 0.3 | 102.3 ± 1.4 | 1.4 |
| #5 | 2.9 ± 0.1 | 6.6 ± 0.1 | 13.7 ± 0.1 | 19.4 ± 0.3 | 97.2 ± 1.5 | 1.5 |
| #6 | 3.1 ± 0.0 | 6.9 ± 0.1 | 14.1 ± 0.0 | 19.0 ± 0.1 | 95.0 ± 0.6 | 0.6 |
| #7 | 2.6 ± 0.2 | 5.8 ± 0.4 | 12.7 ± 0.5 | 18.9 ± 0.3 | 94.6 ± 1.6 | 1.7 |
| #8 | 2.4 ± 0.0 | 5.4 ± 0.0 | 11.1 ± 0.0 | 19.6 ± 0.3 | 98.0 ± 1.5 | 1.5 |

Example 3. Analysis by scanning electron microscopy of spray-dried powders according to Example 1 produced with nozzles with different diameter.

The morphology of the powders dried as described in Example 1 was studied by scanning electron microscopy (SEM) using a SUPRA 40 instrument (Carl Zeiss, Germany). Each powder sample (10 mg) was placed on a sample holder previously covered by a conductive carbon bi-adhesive to allow charge dispersion. The excess particles were removed by a gentle nitrogen flow.

The samples were analyzed in high vacuum conditions at 2.75 10⁻⁶ Torr and the images were collected at 5000× magnification using an accelerating voltage of 1.0 kV.

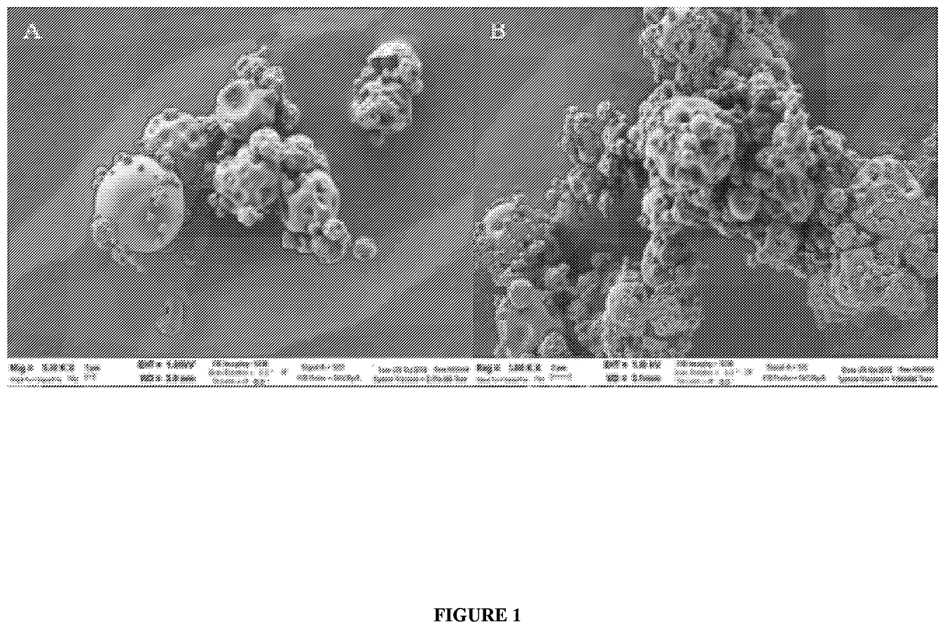

FIG. 1 shows the SEM images of the dried powders produced with a nozzle with 2 or 1.4 mm diameter.

It can be noticed that, in both cases, the composite HA-LANE powders were characterized by rounded particles prevalently with a collapsed surface and in few cases with a swollen and smooth surface. Anyway, the powder produced with a 1.4 mm nozzle diameter showed a great number of smaller particles attached to bigger particles with respect to the one produced with a 2 mm nozzle diameter wherein the number of smaller particles was lower.

Example 4. X-ray diffraction of powders.

The X-ray diffraction of powders analysis was performed on two powders produced with two different HAs:
Prymalhyal 50 (20-50 kDa, Givaudan, France);
PLUS-PH 100 kDa Ph.Eur. grade (Altergon, Italy).

With both polymers a powder containing lidocaine hydrochloride 20% w/w and HA 80% w/w was produced.

The powders were produced starting from a solution containing overall 3% w/v of solutes using a Buchi B290 apparatus using the following process parameters:

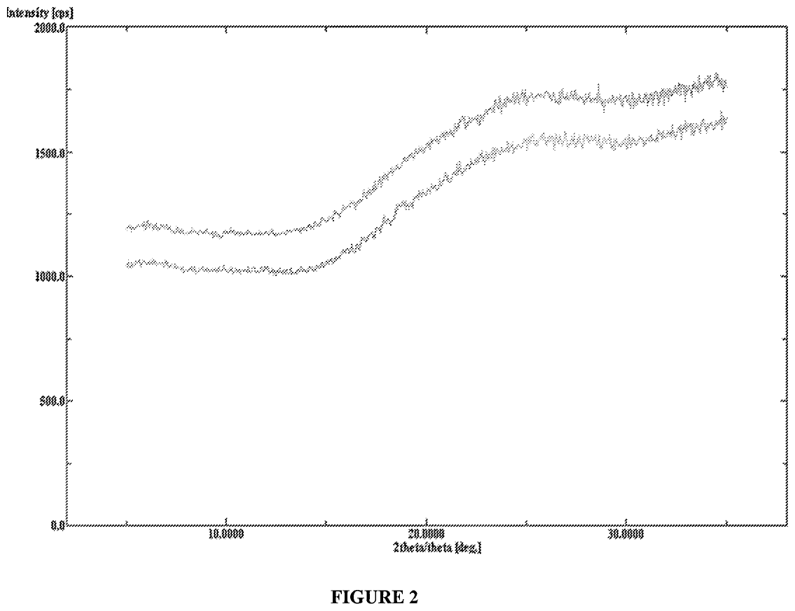

Nozzle diameter (2 mm); supply flow rate (2 mL/min), gas flow rate (600 L/h) input temperature (130° C.), output temperature (70° C.), aspiration flow rate (35 m³/h). The X-ray diffraction of these powders was recorded using a Miniflex diffractometer (Rigaku, Japan) using a radiation of Cu Kα 30 kV, with a scanning speed of 0.05°/minute and a scanning field (2θ) from 5 to 35°. The diffraction patterns (FIG. 2) showed that both powders were amorphous.

Example 5. Aerodynamic size distribution of the particles determined by impact procedure of spray-dried powders containing lidocaine hydrochloride and HA (20-50 kDa).

The composite spray-dried powder contained lidocaine hydrochloride 20% w/w and HA 20-50 kDa 80% w/w.

The components were dissolved separately in water. The two solutions were then combined to obtain a final solution to be dried with a solute content equal to 3% w/v.

Two powders were produced using a Büchi B290 spray-dryer using nozzle with 2.0 or 1.4 mm diameter, respectively. The following process parameters were used: supply flow rate (4 mL/min); gas flow rate 600 L/h; input temperature 130° C.; output temperature (70° C.); aspiration flow 35 m³/h.

Each formulation was loaded onto hypromellose (hydroxypropyl methyl cellulose) capsules Quali-V-I format #3 (Qualicaps, Spain) and aerosolized by means of a dry powder inhaler with a medium-high resistance RS01 (Plastiape, Italy). Each capsule was loaded with 40±0.1 mg of powder corresponding to a lidocaine nominal dose of 8 mg. The particle aerodynamic size was assessed using a Next Generation Impactor (NGI, Copley Scientific, UK) equipped with an induction port (IP) as described in the American Pharmacopoeia, USP. Each determination was performed discharging the content of 4 capsules at 60 L/min sampling rate for 4 seconds so that 4 L of air were aspired through the equipment according to what recommended from the European Pharmacopoeia 10^{th} Edition 2.9.18.

At the end of the deposition experiment, the NGI was disassembled and the quantification of the lidocaine in the deposited powders in each stage was performed using a HPLC validated method.

The lidocaine hydrochloride deposited on each stage of the impactor was recovered with water:methanol (25:75 v/v) aliquots, which were finally transferred to volumetric flasks of suitable volume and brought up to volume with the same solvent mixture. The obtained solutions were filtered through a cellulose acetate syringe filter (0.45 μm porosity and 2.5 cm diameter, GVS Filter Technology, USA) before being injected into HPLC. A volumetric flask was used to collect the powder remained in the RS01® device and in the capsules which were dissolved in ultrapure water at the end of the experiment to verify the complete recovery of the active ingredient.

The delivered dose was determined also using the DUSA (Dose Unit Spray Apparatus) methodology. The delivered lidocaine dose was collected from 10 separate capsules. The capsule content was aerosolized using the RS01® device at 60 L/min and the sampled air volume was equal to 2.0 L. All powders were tested in triplicate.

The dosed lidocaine amount, the delivered dose, the fine particle dose, the fine particle fraction, the median mass aerodynamic diameter (MMAD), and the geometric standard deviation (GSD) for each measurement performed with the impactor were calculated in compliance with the European Pharmacopoeia (10^{th} edition 2.9.18).

The aerodynamic performance of the two powders was assessed calculating:
the emitted dose (ED), obtained as sum of the drug portions recovered from the induction port and all stages of NGI expressed in mg, and the percentage thereof with respect to the nominal dose
the fine particle dose (FPD), namely the drug amount contained in particles with a diameter less than 5 μm, calculated by interpolation according to the European Pharmacopoeia and expressed in mg
the fine particle fraction (FPF), calculated as the ratio between FPD and ED expressed in percentage
the not-breathable particle dose (NB-PD), namely the drug amount contained in particles with aerodynamic diameter higher than 5 μm, obtained as the difference between the emitted dose in the NGI and the fine particle dose, expressed in mg
the not-breathable particle fraction (NB-PF), calculated as the ratio between the not-breathable particle dose and the ED expressed in percentage.

Herein, the term “dose” means the active ingredient amount delivered by a single activation of the inhaler.

The aerosolization performance of the two powders are summarized in Table 3.

The average values of the delivered dose for the spray-dried powders obtained with the nozzles of both diameters determined by NGI measurement, were comprised between 88 and 95% of the dosed amount value (lidocaine amount recovered in the whole impactor summed to that recovered in the capsule and in the device), while, using DUSA methodology they were >97% w/w.

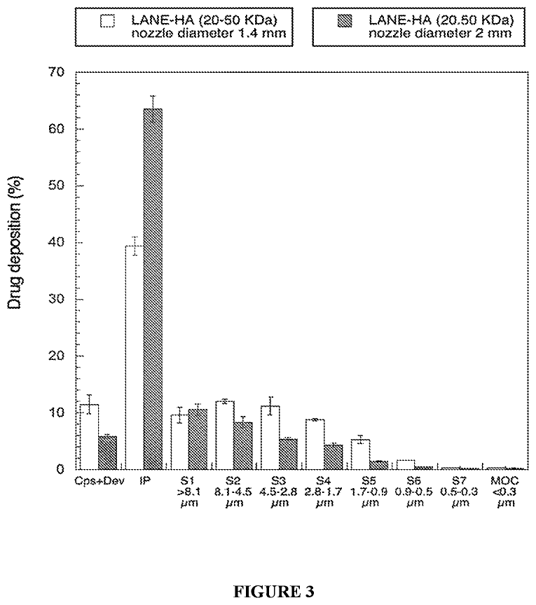

The difference in the fine particle fraction and not-breathable particle fraction was a direct consequence of the different particle size (aerodynamic) distributions of the two powders as highlighted also from the MMAD values and the drug amounts contained in the particles deposited in the various stages in the NGI apparatus, in the induction port (IP) and remained in the RS01 device or in the capsule. In FIG. 3 the drug distribution in the NGI stages of the two powders produced with different diameter nozzles, 1.40 (blank histogram) and 2.00 mm (full histogram) respectively, is reported. The bars represent the standard deviation (n=3).

TABLE 3
Aerodynamic parameters of the two powders containing
8 mg of lidocaine produced with different diameter
nozzles. Average values ± standard deviation (n = 3).

| | Nozzle | Nozzle |
|---|---|---|
| | diameter | diameter |
| Parameters | 1.40 mm | 2.00 mm |
| Emitted dose (mg) DUSA | 7.78 ± 0.08 | 7.85 ± 0.10 |
| Dosed amount (mg) | 7.46 ± 0.09 | 7.36 ± 0.03 |
| Emitted dose (mg) NGI | 6.60 ± 0.05 | 6.93 ± 0.01 |
| Fine particle dose ≤5 μm (mg) | 2.24 ± 0.08 | 1.05 ± 0.09 |
| Fine particle fraction ≤5 μm (%) | 33.93 ± 1.41 | 15.12 ± 1.23 |
| Not-breathable particle | 4.36 ± 0.12 | 5.89 ± 0.08 |
| dose >5 μm (mg) | | |
| Not-breathable particle | 66.07 ± 1.41 | 84.88 ± 1.23 |
| fraction >5 μm (%) | | |
| MMAD (mm) | 3.94 ± 0.11 | 5.60 ± 0.12 |
| GSD | 2.36 ± 0.16 | 2.54 ± 0.05 |

Example 6. Comparison between the dissolution of spray-dried powders and lidocaine.

The in vitro dissolution tests were performed on lidocaine powder raw material, spray-dried HA-lidocaine powders, produced using PLUS-PH 100 kDa Ph.Eur (Altergon, IT, batch nr. 1000008976), Prymalhyal 50 (20-50 kDa, Givaudan, France) and Contipro Biotech (Tech. Grade, 750-1000 kDa, Czech Republic) as bioadhesive polymer through a spray-dryer (Buchi) and using the following process parameters: nozzle diameter (2 mm), flow (2 mL/min), air flow rate (600 L/h), input temperature (130° C.), output temperature (70° C.), and aspiration flow (35 m³/h).

The spray-dried powders had a 3% w/v solid content and contained:
lidocaine hydrochloride 20% (w/w) and HA PLUS-PH 100 kDa 80% (w/w)
lidocaine hydrochloride 60% (w/w) and HA Prymalhyal 50 (20-50 kDa) 40% (w/w)
lidocaine hydrochloride 20% (w/w) and HA Contipro Biotech (750-1000 kDa) 80% (w/w).

The in vitro dissolution tests were performed to compare the powder performance, using RespiCell™ (Sonvico, F et al. Pharmaceutics 2021, 13, 1541), a vertical diffusion cell comprising a 170 cm³ reservoir, filled with the dissolution medium, and a 10 cm length side arm.

The apparatus is consisting of an upper part, the donor chamber, and a lower part, the receiving chamber, held together by means of a metal clamp, but separated by a glass microfiber filter, used as a diffusion membrane, and placed horizontally in contact with the dissolution medium. The receiving chamber contains a magnetic stirrer.

The dissolution medium used for the analysis is phosphate buffered saline (PBS), prepared weighing 8 g of NaCl, 0.2 g of KCl, 1.44 g of Na₂HPO₄, and 0.12 g of KH₂PO₄ dissolved in 1 L of distilled water, with a final pH of 7.4. During the analysis, RespiCell™ was thermostated (Lauda eco silver E4, DE) at 37±0.5° C.

The receiving chamber was filled with PBS and sampled at pre-established time intervals through the side arm of the cell. Before the analysis 1 mL of the dissolution medium was applied on the filter, to get it completely wet.

At the beginning of the analysis, about 8 mg of exactly weighted lidocaine, 40 mg of exactly weighted spray-dried lidocaine-HA PLUS-PH 100 kDa Ph.Eur powder (corresponding to about 8 mg of lidocaine), 13.3 mg of exactly weighted spray-dried lidocaine-HA Prymalhyal 50 powder (corresponding to about 8 mg of lidocaine), or 40 mg of exactly weighted spray-dried lidocaine-HA Contipro Biotech powder (corresponding to about 8 mg of lidocaine), were spread manually on the wet filter and, at pre-established time intervals, 1 mL of the receiving solution was withdrawn from the receiving chamber by means of the side arm and substituted with 1 mL of fresh PBS after each withdrawal to maintain a constant volume of liquid inside the cell.

To assess the not dissolved or trapped in the filter drug amount, the not dissolved residual powder was recovered washing the filter with 5 mL of a methanol:water 75:25 v/v mixture, at the end of the experiment. The drug amount in the samples was quantified by HPLC analysis.

Data were expressed as percentage of dissolved lidocaine; 100% dissolution corresponds to the total drug amount recovered at the end of the experiment (in the cell receiving chamber plus the amount on the filter). The total dissolution time was: 25 minutes for the LANE powder raw material and 135 minutes for the spray-dried lidocaine-HA powders.

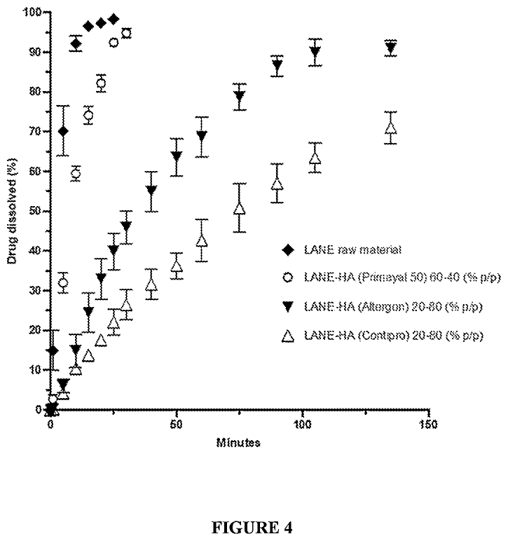

FIG. 4 shows the dissolution profile of the lidocaine raw material and the spray-dried lidocaine-HA powders.

The obtained results showed a different dissolution profile of the LANE raw material and lidocaine-HA powders.

In particular, the dissolution of the spray-dried lidocaine-HA powders is significatively slower: in the case of the lidocaine powder raw material, the total amount of lidocaine was completely dissolved after 15 minutes, while the LANE amount in the spray-dried powder with HA with PLUS-PH 100 kDa, was completely dissolved after about 2 hours. Anyway, the spray-dried powder containing Prymalhyal 50, having a 60% LANE content is significantly faster than the previous one, and dissolved completely after 25 minutes in the dissolution medium.

The dissolution of the spray-dried powder containing HA Contipro Biotech is the slowest, and after 2 hours only 70% of the LANE was dissolved in the dissolution medium.

Table 4 summarizes the dissolved lidocaine amount, the lidocaine amount retained on and inside the filter, and the mass balance (total recovery) of the experiment.

TABLE 4
lidocaine amount recovered from the
dissolution test (about 8 mg of LANE)

| | | Lidocaine | |
|---|---|---|---|
| | | retained | |
| | | on and | |
| | Dissolved | inside the | Total |
| | lidocaine | filter | recovery |
| | (mg) | (mg) | (%) |
| Lidocaine powder raw | 7.9 ± 0.4 | 0.1 ± 0.0 | 94.0 ± 3.3 |
| material | | | |
| Spray-dried powder of | 8.4 ± 0.2 | 0.8 ± 0.2 | 98.6 ± 2.4 |
| lidocaine-HA PLUS-PH 100 | | | |
| kDa | | | |
| Spray-dried powder of | 7.7 ± 0.0 | 0.4 ± 0.1 | 95.7 ± 0.5 |
| lidocaine-HA Prymalhyal 50 | | | |
| Spray-dried powder of | 6.7 ± 0.1 | 1.9 ± 0.4 | 83.7 ± 1.5 |
| lidocaine-HA Contipro | | | |
| Biotech | | | |

Example 7: Characterization of the mixture LANE-Sodium hyaluronate Contipro SD 20:80 by optical microscopy

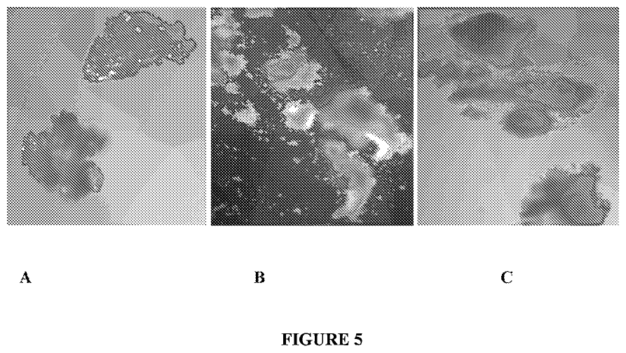

The LANE-HA powders produced according to Example 6 were observed by polarized light microscopy (Optiphor2-POL, Nikon, Japan) to highlight possible birefringence phenomena related to the presence of lidocaine microcrystals in the particle structure. From the obtained images, it was observed that the amorphous particles, HA Contipro Biotech (750-1000 kDa) showed on the surface birefringent microcrystalline lidocaine structures. Said crystallization was observed in the powders having a ratio lidocaine:HA equal to 10:90; 20:80 (FIG. 5, Panel A); 25:75; 30:70.

Using a sodium hyaluronate with a lower molecular weight, i.e., the sodium hyaluronate Prymalhyal 50 (20-50 kDa) a completely amorphous powder was obtained at LANE concentrations both of 20 and 60% w/w. FIG. 5 shows images at polarized light optical microscope (magnification 10×) of particles obtained by spray-drying according to what reported in Example 6 of: LANE:HA (Contipro) 20:80 w/w (Panel A); LANE:HA (Prymalhyal) 20:80 w/w (Panel B), and LANE:HA (Prymalhyal) 60:40 w/w (Panel C).

Example 8. Stability study of spray-dried HA-lidocaine hydrochloride powders.

The stability studies were performed on two spray-dried powders containing lidocaine hydrochloride 20% w/w and two different types of HA: Prymalhyal 50 (20-50 kDa,) and PLUS-PH 100 kDa Ph.Eur produced according to Example 4.

The powders were stored in hypromellose capsules format #3, sealed in aluminum bags, and analyzed after 1 month and 4 months storage in two different conditions:
25° C.—60% relative humidity
40° C.—75% relative humidity.

The chemical stability was assessed quantifying the lidocaine hydrochloride content by HPLC analysis at various storage times.

The physical stability was also assessed by Hot Stage Microscopy (HSM), wherein the samples were observed at polarized light microscope before (T zero) and after the storage (1 and 4 months). The samples were heated from 25° C. to 130° C., with a heating range of 5° C./minute in nitrogen atmosphere. The obtained data are reported in Table 5.

The analysis showed that the powders do not show tendency to develop crystalline portions even after heating, both freshly prepared and at the two different storage times.

TABLE 5
Lidocaine content and particle structure of powders
prepared with different molecular weight HAs and
stored in different storage conditions.

| | | | Lidocaine | Lidocaine | |
|---|---|---|---|---|---|
| | | Lidocaine | content | content | |
| HA | | content | at 1 | at 4 | Structure |
| Mw | Storage | at T0 | month | months | at 4 |
| (kDa) | conditions | (w/w) | (w/w) | (w/w) | months |
| 20-50 | 25° C.-60 | 20.9 ± 0.6 | 19.9 ± 0.3 | 20.5 ± 0.8 | amorphous |
| | RH % | | | | |
| 100 | 40° C.-75 | 20.9 ± 0.6 | 21.2 ± 0.6 | 20.8 ± 0.2 | amorphous |
| | RH % | | | | |

Example 9. Tussigenic challenge test.

The tussigenic challenge test (Fontana, GA, et al. Eur Respir J 1997; 10: 983-98; Lavorini, F et al. Am J Respir Crit Care Med 2001; 163: 1117-1120) was performed on 10 healthy volunteers (8 males, aged comprised between 35 and 70 years) before and after the inhalation of a placebo powder and of a lidocaine-HA powder prepared according to Example 4 with sodium hyaluronate PLUS-PH (100 kDa).

Cough was induced by the inhalation of ultrasonically nebulized distilled water (fog) produced by the ultrasonic nebulizer Mist-O2-Gen (EN143A Model, Timeter, PA, USA). It is reported in the literature that the mass median aerodynamic diameter of the aerosol droplets generated by the nebulizer is 3.6-5.7 μm (Phipps, PR, et al. Chest 1990; 7 1327-1332). The nebulizer reservoir was filled with 180 mL of distillated water; the output of the aerosol was adjusted by a potentiometer and monitored as a direct current (DC) signal on an oscilloscope. The output can be progressively increased for levels corresponding to 5% of the maximum attainable output level.

The fog was inhaled by the volunteers during the normal breathing at rest and the inhalation time for each concentration was standardized to 1 minute. 2-3 rest minutes were scheduled between each inhalation of fog. The nebulizer output range used in the experiments could vary from 30 to 100% of the maximum DC signal and the corresponding nebulized water amount (average values) ranged from 0.08 to 4.45 mL/minute.

The cough onset was revealed recording the expiratory flow by means of a pneumotachograph Fleish type nr. 4. After appearance of cough, the test was stopped, and the subjects were allowed to rest for 30 min. Then, the test was resumed with the inhalation of the fog corresponding to an output value immediately lower to the latter administered. If the cough could be provoked again at the same fog level which was previously able to evocate cough, the challenge was stopped, and the level of fog taken as the subject's cough threshold (T). On the contrary, if a cough response was not obtained, the test was resumed and continued until cough was provoked twice with the same fog level. Therefore, the lower fog level able to evoke at least a cough during two consecutive tests, separated by a 30 minutes interval, was taken as the cough threshold.

During the inhalation of each nebulizer output, the intensity of the urge-to-cough (UTC) was assessed by a 10-cm visual analog scale (VAS) (Lavorini F, et al. Am J Respir Crit Care Med 2006; 176: 825-32). The extremes of the VAS (i.e., “no desire to cough” and “extreme urge to cough”) were displayed on the two ends of a display placed in front of the subject. The “extreme urge to cough” was explained to each participant as a need to cough which is impossible to resist. The display was connected to a linear potentiometer equipped with a cursor by which the subject could quantify the UTC level. Both the display and the potentiometer were 10 cm long. Equal distances on the display and potentiometer represented equal variation in the intensity of UTC.

After evaluating the cough threshold, the subject, in days separated from 24-48 hours, were administered with placebo (lactose) or HA-lidocaine powder using the RS01 DPI. The subjects were carefully instructed by trained medical personnel to inhale as fast as possible starting from the level near to the residual volume up to the total lung capacity, subsequently holding the breath for about 10 seconds. For each subject the capsule was inhaled twice to minimize possible dry powder residue. After about 5 minutes, the tussigenic challenge was repeated with the same modalities than the baseline test. If the subject did not cough, he/she was subjected to fog inhalation equal to 1.3×T, 1.6×T and equal to 100% of the nebulizer output. The time required to re-establish the baseline (i.e. predrug) cough threshold of the subject was calculated as well.

The placebo inhalation did not affect the cough threshold and the corresponding UTC. On the contrary, the HA-lidocaine powder increased significantly the cough threshold and UTC in the 10 tested subjects with a median increase equal to 2.13 times. The duration of the effect was about 50±8 min.

1. Pharmaceutical compositions in the form of a dry powder for inhalation comprising a local anesthetic and a hydrophilic biocompatible polymer.

2. Compositions according to claim 1 wherein the local anesthetic is selected from procaine, chloroprocaine, lidocaine, prilocaine, mepivacaine, bupivacaine, etidocaine, ropivacaine, and tetracaine and/or salts and/or solvates thereof.

3. Compositions according to claim 2 wherein the local anesthetic is lidocaine.

4. Compositions according to claim 1, wherein the hydrophilic biocompatible polymer is selected from hyaluronic acid salts, water-soluble cellulose derivatives, polyethylene glycols, polyvinyl pyrrolidones, polyvinyl alcohols and/or mixtures thereof.

5. Compositions according to claim 4 wherein the hydrophilic biocompatible polymer is sodium hyaluronate.

6. Compositions according to claim 1, wherein the dry powder is consisting of spray-dried micro-particles having a defined size comprised between 2 and 18 microns, a mass median aerodynamic diameter comprised between 4.0 and 6.0 microns, and a fine particle fraction not higher than 35% by mass.

7. Compositions according to claim 6 wherein the particle size distribution of the micro-particles is characterized by d(0.1) comprised between 2.0 and 5.0 microns, d(0.5) comprised between 5.0 and 9.0 microns, and d(0.9) comprised between 11 and 18 microns.

8. A process for preparing the micro-particles of claim 6, comprising the following steps:
i) selecting a hydrophilic biocompatible polymer and dissolving said hydrophilic biocompatible polymer dissolution thereof in a suitable solvent at a suitable concentration;
ii) selecting a local anaesthetic (LANE) drug and dissolving said LANE drug in water at a suitable concentration;
iii) adding the solution of step ii) to the solution of step i) keeping under stirring the resulting solution;
iv) spray-drying the solution of step iii);
v) collecting the resulting powder in the form of particles; and
vi) optional micronizing said particles.

9. The process according to claim 8 wherein in step iv) a spray-dryer nozzle having a diameter comprised between 0.7 mm and 3 mm is used.

11. (canceled)

12. Method of treating cough in a subject in need thereof with the composition of claim 1, said method comprising:
exposing said subject to a pharmaceutical effective amount of the compositions of claim 1 via inhalation and
treating said cough in said subject.
